(19) United States
(12) Patent Application Publication
(10) Pub. No.: US 2022/0202610 A1
(43) Pub. Date: Jun. 30, 2022

(54) CONTACT LENS WITH SUSTAINED RELEASE PROPERTY

(71) Applicant: Industrial Technology Research Institute, Hsinchu (TW)

(72) Inventors: Felice CHENG, Zhubei City (TW); Wen-Chia HUANG, Taichung City (TW); Shao-Chan YIN, Hsinchu City (TW); Po-Hsien KUO, Tainan City (TW); Li-Wen CHANG, New Taipei City (TW); Yen-Jen WANG, Taichung City (TW)

(73) Assignee: Industrial Technology Research Institute, Hsinchu (TW)

(21) Appl. No.: 17/139,360

(22) Filed: Dec. 31, 2020

(51) Int. Cl. A61F 9/00; G02B 1/04; C08B 37/16; C08G 83/00; C08F 220/18; C08F 220/20; C08F 222/10

(52) U.S. Cl. CPC A61F 9/0017; G02B 1/043; C08B 37/0015; C08F 222/102; C08F 220/1802; C08F 220/20; C08G 83/007

(57) ABSTRACT

A contact lens with sustained-release property is provided. The contact lens includes a polymer, pseudorotaxane embedded in the polymer, and a stabilizer embedded in the pseudorotaxane. The polymer is polymerized by monomer materials. The monomer materials include ethyl methacrylate, ethylene glycol dimethacrylate, methacrylic acid, 2-hydroxyethyl methacrylate, or a combination thereof. The stabilizer includes surfactant, amino acid with a polar side chain, oligopeptide containing at least one amino acid with a polar side chain, purine or its derivative, or a combination thereof.

### TECHNICAL FIELD

The present disclosure relates to a contact lens, and more particularly to a contact lens with sustained-release property.

### BACKGROUND

The contact lens market is an oligopolistic market. More than 90% of the market sales is exclusively occupied by the top four contact lens manufacturers, including Bausch & Lomb, Johnson & Johnson, Ciba Vision, and Cooper Vision. If Taiwanese manufacturers do not produce contact lens products with special functionality, they can only do low gross margin foundry work or cut prices for sales.

### SUMMARY

In accordance with one embodiment of the present disclosure, a contact lens with a sustained-release property is provided. The contact lens includes a polymer polymerized by monomer materials, wherein the monomer materials include ethyl methacrylate, ethylene glycol dimethacrylate, methacrylic acid, 2-hydroxyethyl methacrylate, or a combination thereof; pseudorotaxane embedded in the polymer; and a stabilizer embedded in the pseudorotaxane, wherein the stabilizer includes surfactants, amino acids with a polar side chain, oligopeptides containing at least one amino acid with a polar side chain, purine or its derivatives, or a combination thereof.

In accordance with one embodiment of the present disclosure, a contact lens used for sustained release of drugs is provided. The contact lens includes: a polymer polymerized by monomer materials, wherein the monomer materials include ethyl methacrylate, ethylene glycol dimethacrylate, methacrylic acid, 2-hydroxyethyl methacrylate, or a combination thereof; pseudorotaxane embedded in the polymer; and a stabilizer embedded in the pseudorotaxane, wherein the stabilizer includes surfactants, amino acids with a polar side chain, oligopeptides containing at least one amino acid with a polar side chain, purine or its derivatives, or a combination thereof.

A detailed description is given in the following embodiments with reference to the accompanying drawings.

### BRIEF DESCRIPTION OF THE DRAWINGS

FIG. 1 shows the release of Menthol from each lens over time in accordance with one embodiment of the present disclosure;

FIG. 2 shows the changes in tear secretion of rabbit eyes worn with contact lenses with increasing wearing time in accordance with one embodiment of the present disclosure;

FIG. 3 shows the comparison of the differences in tear secretion before and after wearing lenses on rabbit eyes in accordance with one embodiment of the present disclosure;

FIG. 4 shows the release of Dexamethasone (DEX) from each lens over time in accordance with one embodiment of the present disclosure;

FIG. 5 shows the drug loading of Dexamethasone (DEX) of each lens in accordance with one embodiment of the present disclosure;

FIG. 6 shows the release of Sunitinib malate (SM) from each lens over time in accordance with one embodiment of the present disclosure; and

FIG. 7 shows the drug loading of Sunitinib malate (SM) of each lens in accordance with one embodiment of the present disclosure.

### DETAILED DESCRIPTION

In accordance with one embodiment of the present disclosure, a contact lens with a sustained-release property is provided. The contact lens includes: a polymer polymerized by monomer materials, wherein the monomer materials include ethyl methacrylate (EMA), ethylene glycol dimethacrylate (EGDMA), methacrylic acid (MAA), 2-hydroxyethyl methacrylate (HEMA), or a combination thereof; pseudorotaxane embedded in the polymer; and a stabilizer embedded in the pseudorotaxane, wherein the stabilizer includes surfactants, amino acids with a polar side chain, oligopeptides containing at least one amino acid with a polar side chain, purine or its derivatives, or a combination thereof, but not limited thereto. The pseudorotaxane formed by cyclodextrin and polyethylene glycol (PEG) does not have a reactive double bond (C═C) structure and will not participate in the polymerization reaction forming contact lens hydrogels. Since a cross-linking agent is added when the contact lens is polymerized, the contact lens hydrogel will form a three-dimensional network cross-linked structure, and the pseudorotaxane is dispersed in the voids of the three-dimensional network structure. The stabilizer can be adsorbed on the cyclodextrin of the pseudorotaxane by forming intermolecular hydrogen bonds with the cyclodextrin.

In some embodiments, the pseudorotaxane may be formed by cyclodextrin (CD) and polyethylene glycol (PEG), but the present disclosure is not limited thereto. In one embodiment of the present disclosure, the pseudorotaxane does not have a capping group and can be easily formed by combining general commercial ophthalmic excipients such as polyethylene glycol and cyclodextrin. In some embodiments, the cyclodextrin may include α-cyclodextrin, β-cyclodextrin, γ-cyclodextrin, hydroxypropyl-alpha-cyclodextrin, hydroxypropyl-beta-cyclodextrin, hydroxypropyl-gamma-cyclodextrin, sulfobutyl-beta-cyclodextrin, or other suitable cyclodextrin derivatives, but not limited thereto. In one embodiment of the present disclosure, the cyclodextrin used is a commercial cyclodextrin with a hydroxypropyl group, and has not been modified by chemical modification. The cyclodextrin does not have a double bond structure (C═C) and therefore does not participate in the polymerization of contact lenses. In some embodiments, the average molecular weight of the polyethylene glycol is ranging between about 2,000 and about 20,000, for example, between about 4,000 and about 10,000, or about 6,000 etc., but not limited thereto. When the molecular weight of the polyethylene glycol is less than 20,000, it is easy to dissolve into the monomer mixture during casting of contact lens. In one embodiment of the present disclosure, the polyethylene glycol used is a general polyethylene glycol without modification with capping groups at the two ends, which is an ophthalmic excipient allowed by the FDA. In some embodiments, the weight ratio of the cyclodextrin to the polyethylene glycol is ranging between about 0.5:1 and about 500:1, for example, between about 10:1 and about 100:1, or between about 1:1 and about 20:1, but not limited thereto. In some embodiments, the weight ratio of the cyclodextrin to the polyethylene glycol is about 185:11, but not limited thereto.

In some embodiments, the weight ratio of the pseudorotaxane to the polymer is ranging between about 1:4 and about 1:200, for example, between about 1:10 and about 1:100, or between about 1:20 and 1:40, etc., but not limited thereto.

In some embodiments, the stabilizer may include surfactants, amino acids with a polar side chain, oligopeptides containing at least one amino acid with a polar side chain, purine or its derivatives, or a combination thereof, but not limited thereto. The surfactant may include, but not limited thereto, Tween 80, Tween 20 Span 80, DSPE-PEG, DSPE-PEG derivatives, or a combination thereof. In one embodiment, the surfactant may be Tween 80.

The amino acid with a polar side chain merely needs to be an amino acid with a polar side chain, and it can be a natural amino acid or a non-natural amino acid without limitation. For example, the amino acid with a polar side chain that can be used as the stabilizer may include cysteine, glutamine, glutamic acid, histidine, or a combination thereof, but not limited thereto.

In addition, the oligopeptide containing at least one amino acid with a polar side chain has no special restriction, as long as it contains at least one amino acid with a polar side chain in the amino acid composition. For example, the oligopeptide containing at least one amino acid with a polar side chain may have only one amino acid with a polar side chain, or may have several amino acids with a polar side chain, or the oligopeptide containing at least one amino acid with a polar side chain may also be composed of amino acids with a polar side chain. Furthermore, in the oligopeptide containing at least one amino acid with a polar side chain, each amino acid may independently be any amino acid, as long as it contains at least one amino acid with a polar side chain in the amino acid composition. In addition, in the oligopeptide containing at least one amino acid with a polar side chain, the position of the at least one amino acid with a polar side chain in the oligopeptide is not particularly limited, and it may be independently at any position in the oligopeptide. The at least one amino acid with a polar side chain in the oligopeptide may independently include cysteine, glutamine, glutamic acid, histidine, or a combination thereof, etc., but not limited thereto. In one embodiment, the oligopeptide containing at least one amino acid with a polar side chain may have about 2-8 amino acids, for example, 2-3, 2-6, 2, 3, 4, 5, 6, 7 or 8 amino acids, etc., but not limited thereto. In addition, the examples of the oligopeptide containing at least one amino acid with a polar side chain may include carnosine, glutathione (GSH), or leucine-glycine-glycine (Leu-Gly-Gly), etc., but not limited thereto.

In addition, the examples of the purine may include adenine, guanine, or a combination thereof, etc., but not limited thereto. The purine derivatives may include, but not limited thereto, caffeine, theobromine, isoguanine, xanthine, hypoxanthine, uric acid, or a combination thereof, etc. In one embodiment, the purine derivatives that can be used as the stabilizer is caffeine.

In some embodiments, the weight ratio of the stabilizer to the pseudorotaxane is ranging between about 1:1 and about 1:20, for example, between about 1:1 and about 1:10, etc., but not limited thereto.

In some embodiments, the contact lens with a sustained-release property of the present disclosure further includes an active ingredient which is embedded in the pseudorotaxane. In some embodiments, the active ingredient may include menthol, steroids, steroid derivatives, antibiotics, tyrosine kinase inhibitors, or a combination thereof, but the present disclosure is not limited thereto. In some embodiments, the weight ratio of the active ingredient to the pseudorotaxane is ranging between about 1:0.1 and about 1:40,000, for example, between about 1:0.5 and about 1:6,000, between about 1:10 and about 1:1,000, between about 1:1 and about 1:10, or between about 1:2.3 and about 1:5.75, etc., but not limited thereto. In some embodiments, the content of the active ingredient in the contact lens may be ranging between about 0.1 μg/lens and about 1,000 μg/lens, for example, between about 0.1 μg/lens and about 500 μg/lens, or between about 100 μg/lens and about 250 μg/lens, etc., but not limited thereto.

In some embodiments, the contact lens is further placed in a preservation solution. In some embodiments, the preservation solution provides the stabilizer embedded in the pseudorotaxane. In some embodiments, the concentration of the stabilizer in the preservation solution is ranging between about 0.01 wt % and about 2 wt %. In one embodiment of the present disclosure, the preservation solution used is a general preservation solution for contact lenses, and its composition is, for example, an isotonic solution containing boric acid, sodium chloride, potassium chloride, and disodium hydrogen phosphate. The stabilizer is placed in the preservation solution so that the stabilizer such as caffeine will not be degraded due to the moist heat sterilization. The stabilizer is placed in the preservation solution before the sterilization, which can effectively maintain the stability of drug loading.

In one embodiment of the present disclosure, cyclodextrin is mixed with polyethylene glycol to form pseudorotaxane. The pseudorotaxane is then mixed with a monomer mixture used for casting contact lens (the monomer mixture including at least ethyl methacrylate, ethylene glycol dimethacrylate, methacrylic acid and hydroxyethyl methacrylate) to cast contact lens to prepare a contact lens drug delivery system. The pseudorotaxane embedded in the contact lens not only improves the adsorption capacity of the contact lens and the active ingredients (especially the hydrophobic active ingredients), but also improves the lubricity of the contact lens. When this type of contact lens is loaded with at least one active ingredient, the drug loaded therein can be released from the contact lens by diffusion to achieve the purpose of drug release.

In one embodiment of the present disclosure, the manufactured high-comfort contact lens is a drug delivery system platform that can be loaded with eye care ingredients, such as menthol, an active ingredient commonly used in over-the-counter eye drops. When the user wears the menthol-loaded contact lens, the active ingredient will be slowly released from the contact lens, which makes the wearer's eyes cool and promotes the secretion of tears, thereby solving the discomfort such as dryness and tingling that is easily caused by wearing contact lenses for a long time, and effectively improving the comfort of contact lenses.

### Preparation Example 1

Preparation of Pseudorotaxane

7.4 g of 2-hydroxypropyl-β-cyclodextrin (HP-β-CD) and 0.44 g of polyethylene glycol (PEG6k, Mw: 6,000 g/mol) were dissolved in 120 mL of deionized water, shaken and homogenized for 1 hour in an ultrasonic water bath, so that 2-hydroxypropyl-β-cyclodextrin and polyethylene glycol were fully dissolved. Next, the solution was concentrated under reduced pressure to remove most of the water, then the sample was freeze-dried (−50° C., overnight) to obtain pseudorotaxane powder (the powder is crystalline, easy to scrape and not stick to the spatula).

### Preparation Example 2

Preparation of Dry Contact Lens

The pseudorotaxane powder prepared in Preparation Example 1 and a monomer solution for casting contact lenses (including the ingredients such as ethyl methacrylate (EMA), ethylene glycol dimethacrylate (EGDMA), methacrylic acid (MAA), 2-hydroxyethyl methacrylate (HEMA) and a photoinitiator) were mixed, such that the content of the pseudorotaxane in the lens syrup reached 2.5 wt %, with continue stirring and homogenizing until the pseudorotaxane was completely dissolved. Next, the mixed solution was injected into a contact lens casting mold, and light-polymerization was performed through the cast molding method. After demolding, a dry contact lens was obtained. The photoinitiator used here includes azobisisobutyronitrile, 2,4,6-trimethylbenzoyldiphenyl-phosphineoxide or 2-hydroxy-2-methylpropiophenone, etc., but not limited thereto.

### Preparation Example 3

Preparation of Menthol-Loaded Lenses (Stabilizer: Caffeine)

1. Lens hydration:

(1) The dry lens to be hydrated prepared in Preparation Example 2 was transferred to a hydration extraction tray.

(2) According to the formula (i.e. the formula for calculating the volume of hydrated brine: number of lenses×20 mL), the required volume of the hydrated brine was calculated. The hydrated brine was poured into a 5-L beaker and heat to 85±2° C.

(3) After the thermometer showed 85±2° C., the hydration extraction tray was put into the hydration brine and continuously heated for 90 minutes.

(4) After 90 minutes, the hydration extraction tray (including lenses) was taken out and placed in another empty 5 L beaker.

(5) Hydrated saline with the same volume was add into the beaker, and stood at room temperature for 120 minutes to obtain hydrated lenses.

2. Lens loaded with menthol:

(1) 2,000 ppm menthol (2 mg/mL) and 2 wt % caffeine (20 mg/mL) were prepared and dissolved in saline at 60° C.

(2) The hydrated lens was taken out, dried with a lens cleaning paper, and put in a 5 mL ampoule.

(3) While hot, 1.3 mL of saline solution (containing 2,000 ppm menthol and 2 wt % caffeine) was poured into a 5 mL ampoule containing 1 lens. The ampoule was capped and placed in an autoclave for sterilizing at 121° C. for 30 minutes. The overall procedure was about 2 hours, and the drug-loaded lens was obtained after sterilization.

### Preparation Example 4

Preparation of Menthol-Loaded Lenses (Stabilizer: Tween 80)

1. Lens hydration:

(1) The dry lens to be hydrated prepared in Preparation Example 2 was transferred to a hydration extraction tray.

(2) According to the formula (i.e. the formula for calculating the volume of hydrated brine: number of lenses×20 mL), the required volume of the hydrated brine was calculated. The hydrated brine was poured into a 5 L beaker and heat to 85±2° C.

(3) After the thermometer showed 85±2° C., the hydration extraction tray was put into the hydration brine and continuously heated for 90 minutes.

(4) After 90 minutes, the hydration extraction tray (including lenses) was taken out and placed in another empty 5 L beaker.

(5) Hydrated saline (containing 0.1 wt % Tween 80) with the same volume was add into the beaker, and stood at room temperature for 120 minutes to obtain hydrated lenses.

2. Lens loaded with menthol:

(1) 2,000 ppm menthol was prepared and dissolved in saline at 60° C.

(2) The hydrated lens was taken out, dried with a lens cleaning paper, and put in a 5 mL ampoule.

(3) While hot, 1.3 mL of saline solution (containing 2,000 ppm menthol) was poured into a 5 mL ampoule containing 1 lens. The ampoule was capped and placed in an autoclave for sterilizing at 121° C. for 30 minutes. The overall procedure was about 2 hours, and the drug-loaded lens was obtained after sterilization.

### Example 1

Release of Lens Drug (Menthol)

Experimental design:

1. Sampling time point: 0, 2, 4 and 8 hours (N=3)

2. Test groups:

The lens composition includes ethyl methacrylate, ethylene glycol dimethacrylate, methacrylic acid, hydroxyethyl methacrylate and a small amount of photoinitiator, etc. In this example, the ratio of pseudorotaxane to contact lens casting monomer mixture reaches 2.5 wt %.

First group: P-2.5HPC7/P6k-38CL

Second group: P-2.5HPC7/P6k-38CL+caffeine (Caf)

Third group: P-2.5HPC7/P6k-38CL+Tween 80 (Tw80)

3. Conditions of drug release:

(1) Single drug-loaded lens was placed in a 5 mL ampoule. 1 mL of saline was added as a release medium. The lens was placed in a shake oven (MS Hybridization Shaking Oven, MO-AOR, Major Science), and a drug release test was conducted under the operating parameters of 35° C. and 100 rpm. No release solution was replaced throughout. Sampling: the lens was taken out at each time point. The drug was extracted with ethanol (98 wt %). The drug content was analyzed by HPLC with RP-C18 column. After the lens was taken out, no drug release test was performed.

(2) The extraction method of menthol in the lens: one lens was taken out of the ampoule used for the release test. Lens surface moisture was fully absorbed with a lens cleaning paper. The one lens was immersed in 1 mL of ethanol (98 wt %) to stand for 8 hours to extract the drug. Then the drug content was analyzed by HPLC.

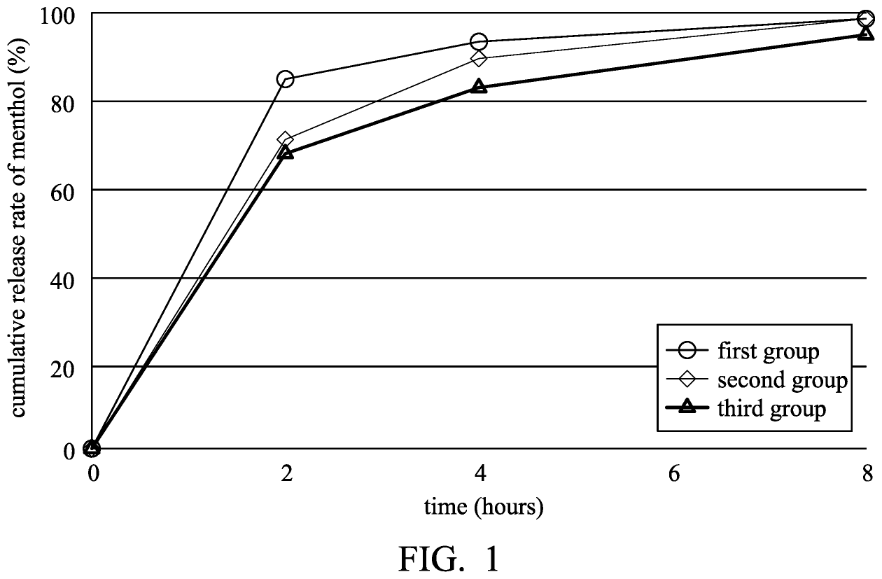

4. Release of lens drug (referring to FIG. 1):

(1) The loading of menthol in the lens is about 175-190 μg/lens. The addition of the stabilizer (i.e. caffeine (second group) and Tween 80 (third group)) has no significant effect on drug loading.

(2) After the moist heat sterilization, most of the menthol in the lens preservation solution escapes.

(3) After the lens is loaded with the stabilizer, the drug release rate is delayed.

### Example 2

The Effect of Menthol-Containing Lenses Worn on Living Animals on Tear Secretion

In this example, the rabbit was worn with contact lenses. The effect of menthol release on tear secretion of rabbit eyes worn with contact lenses was directly evaluated. The test animal used in this example was a 2-2.5 kg male New Zealand white rabbit. Before the test, in order to prevent the nictitating membrane of the rabbit eyes from affecting the wearing of contact lenses, the nictitating membrane was sutured and fixed by a surgical approach. The test is divided into three groups. The test groups are shown in the following Table 1.

TABLE 1

| Test groups | Lenses used | Test operation |
|---|---|---|
| Control group | Jingshuo lenses with | (1) Rabbit eyes were only worn with Jingshuo |
| | 38% moisture content | lenses with 38% moisture content. |
| | | (2) Nictitating membrane fixation rabbits |
| | | model (eyeball = 4): Rabbits were worn |
| | | with commercially available contact |
| | | lenses, and rabbit tear secretion was |
| | | evaluated at 0, 2, 4 and 8 hours |
| | | (Schirmer's test). |
| Eye drops + | Jingshuo lenses with | (1) After the rabbit eyes were worn with |
| Jingshuo lenses | 38% moisture content | Jingshuo lenses with 38% moisture |
| | | content, the eye drops (containing menthol |
| | | aqueous solution) were dropped into |
| | | the rabbit eyes, so that the ocular drug |
| | | exposure reached 330 μg. |
| | | (2) Nictitating membrane fixation rabbits |
| | | model (eyeball = 2): After the rabbit was |
| | | worn with commercially available contact |
| | | lenses, 330 μg of menthol was dropped |
| | | into the rabbit's eyes (four drops of saline |
| | | containing 0.185 wt % menthol, the volume |
| | | of eye drops for four times was |
| | | respectively 50 μL, 50 μL, 50 μL and 27 |
| | | μL, each time 5 minutes apart), and rabbit |
| | | tear secretion was evaluated at 0, 2, 4 and 8 |
| | | hours (Schirmer's test). |
| Menthol-loaded | Menthol-loaded lenses | (1) Rabbit eyes were worn with the |
| lenses | (the contact lenses cast | menthol-loaded lenses. |
| | with 2.5 wt % | (2) Nictitating membrane fixation rabbits |
| | pseudorotaxane was | model (eyeball = 2): The rabbit was worn |
| | used, and tween 80 was | with the menthol-loaded lenses, and rabbit |
| | used as a stabilizer) | tear secretion was evaluated at 0, 2, 4 and 8 |
| | | hours (Schirmer's test). |

Evaluation of Tear Secretion (Schirmer's Test):

The Schirmer paper strip was inserted into conjunctival sac location around the junction of the middle and outer thirds of the lower lid. The rest was hung on the skin surface. The eyes were closed. After 5 minutes, the filter paper was taken out, and the immersed length of the filter paper was measured.

Animal Test Results:

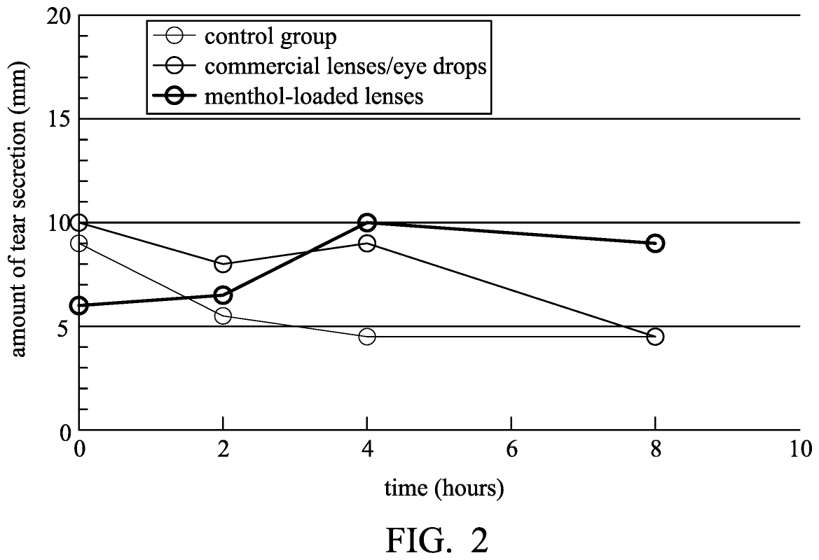

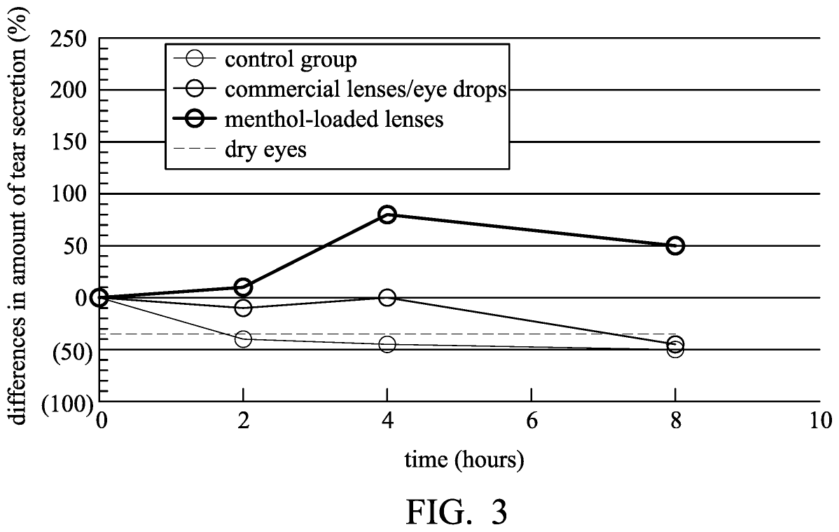

Rabbit eyes were worn with each group of lenses. The secretion of rabbit tears with the time of wearing the lenses is shown in FIGS. 2 and 3. FIG. 2 shows the changes in tear secretion of rabbit eyes worn with contact lenses with increasing wearing time. FIG. 3 shows the comparison of the differences in tear secretion before and after wearing lenses on rabbit eyes.

According to the results in FIGS. 2 and 3, when rabbit eyes wear Jingshuo commercial lenses (with 38% moisture content), the amount of tear secretion decreases with the increase of wearing time. When rabbit eyes wear contact lenses for 8 hours, the amount of tear secretion has dropped to 53% of that before wearing (as shown in FIG. 3). In contrast, when rabbit eyes wear Jingshuo lenses with 38% moisture content, and then the saline containing 0.185 wt % menthol is dropped, the tear secretion can be improved within 4 hours. This should be caused by activating the transient receptor potential melastatin-8 (TRPM8) channel on the cornea by menthol, which induces tear production. However, this effect is not much different from that of the eye drops and cannot last. When rabbit eyes wear the contact lens for 4 hours, the tears from the rabbit's eyes begin to decrease. After 8 hours of wearing the lens, the amount of tear secretion in the rabbit's eyes also drops to 49% of that before wearing the lens (as shown in FIG. 3). Different from the above, when rabbit eyes wear the lenses developed in the present disclosure, the tear secretion of rabbit eyes gradually increases with the increase of wearing time. Even if the lens is worn for 8 hours, the tear secretion of rabbits does not decrease by more than 34% compared with before wearing the lens, and the symptoms of dry eye does not occur (as shown in FIG. 3). It can be proved that if the contact lens developed in the present disclosure is used, the problem of dry eyes caused by wearing contact lenses for a long time can be solved, and the wearing comfort of contact lenses can be greatly improved.

### Preparation Example 5

Preparation of Dexamethasone-Loaded Lenses (Stabilizer: Caffeine)

1. Lens hydration:

(1) The dry lens to be hydrated prepared in Preparation Example 2 was transferred to a hydration extraction tray (a lid was covered to prevent the lens from flowing out).

(2) According to the formula (i.e. the formula for calculating the volume of hydrated brine: number of lenses×20 mL), the required volume of the hydrated brine was calculated. The hydrated brine was poured into a 5 L beaker and heat to 85±2° C.

(3) After the thermometer showed 85±2° C., the hydration extraction tray was put into the hydration brine and continuously heated for 90 minutes.

(4) After 90 minutes, the hydration extraction tray (including lenses) was taken out and placed in another empty 5 L beaker.

(5) Hydrated saline with the same volume was add into the beaker, and stood at room temperature for 120 minutes to obtain hydrated lenses.

2. Lens loaded with dexamethasone:

(1) 150 ppm Dexamethasone was prepared and dissolved in saline at 80° C.

(2) While hot, 1.3 mL of saline solution (containing 150 ppm Dexamethasone) was poured into a 5 mL ampoule containing 1 lens. The lens was immersed for 4 hours at room temperature.

(3) The lens preservation solution was replaced with a saline solution containing 100 ppm Dexamethasone and 0.1 wt % caffeine.

(4) The ampoule was capped and placed in an autoclave for sterilizing at 121° C. for 30 minutes. The overall procedure was about 2 hours, and the drug-loaded lens was obtained after sterilization.

### Example 3

Release of Lens Drug (Dexamethasone)

Experimental design:

1. Sampling time point: 0, 2, 4 and 8 hours (N=3)

2. Test groups:

The lens composition includes ethyl methacrylate, ethylene glycol dimethacrylate, methacrylic acid, hydroxyethyl methacrylate and a small amount of photoinitiator, etc.

First group: P-2.5HPC7/P6k-38CL (P-2.5%-38CL) (the content of the pseudorotaxane was up to 2.5 wt %)

Second group: P-2.5%-38CL+caffeine (P-2.5%-38CL-Caf) (the content of the pseudorotaxane was up to 2.5 wt %)

Third group: P-5HPC7/P6k-38CL (P-5%-38CL) (the content of the pseudorotaxane was up to 5 wt %)

Fourth group: P-5%-38CL+caffeine (P-5%-38CL-Caf) (the content of the pseudorotaxane was up to 5 wt %)

3. Conditions of drug release:

(1) Single drug-loaded lens was placed in a 5 mL ampoule. 1 mL of saline was added as a release medium. The lens was placed in a shake oven (MS Hybridization Shaking Oven, MO-AOR, Major Science), and a drug release test was conducted under the operating parameters of 35° C. and 100 rpm. The drug release solution was replaced with fresh saline every 2 hours.

(2) The drug content in the lens preservation solution and the drug content in the lens were analyzed by HPLC with RP-C18 column. The extraction method of drug (Dexamethasone, DEX) in the lens: one lens was taken out of the preservation solution. Lens surface moisture was fully absorbed with a lens cleaning paper. The one lens was immersed in 1 mL of methanol to stand for 8 hours to extract the drug. Then the drug content was analyzed by HPLC.

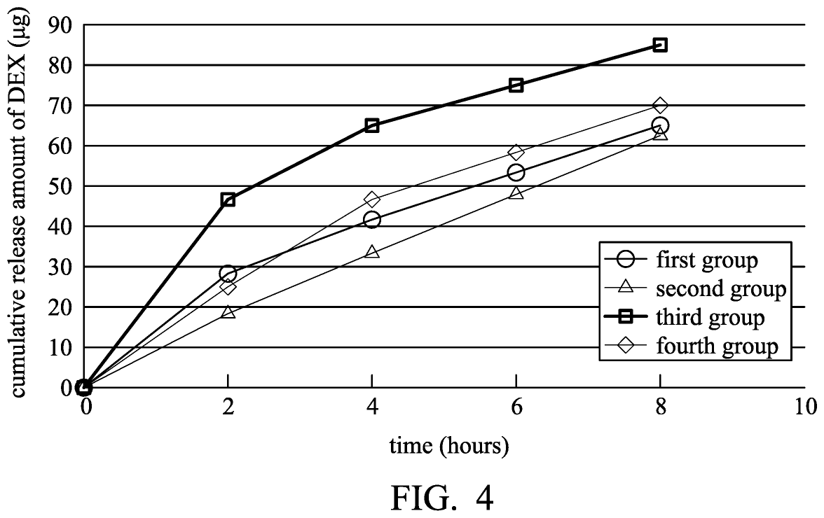

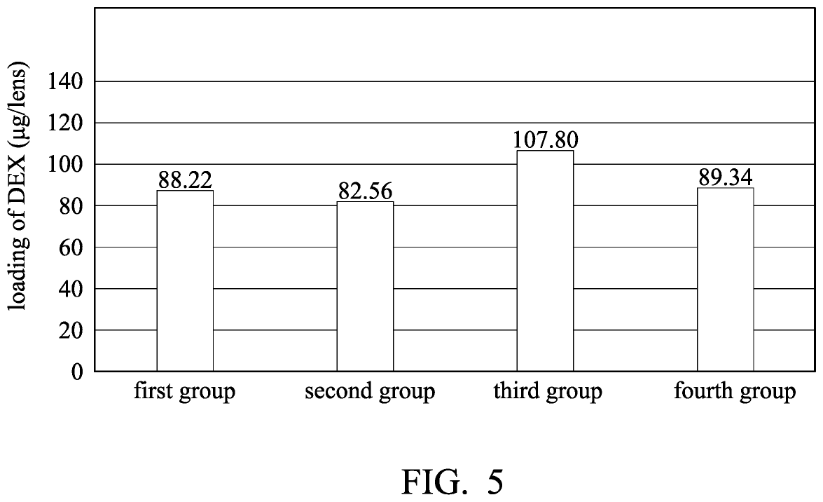

4. Release of lens drug (referring to FIGS. 4 and 5):

(1) The loading of Dexamethasone in the lens is about 80-110 μg/lens. The addition of caffeine (the lens was immersed in 1 mg caffeine/mL) slightly reduces the drug loading of the lens to Dexamethasone (as shown in FIG. 5).

(2) The moist heat sterilization hardly causes the loss of Dexamethasone and caffeine.

(3) The loading of caffeine in the lens is about 155 μg/lens.

(4) After the lens is loaded with caffeine, the drug release rate is delayed (as shown in FIG. 4).

(5) The higher the content of cyclodextrin in the lens, the more obvious the delay of drug release.

### Preparation Example 6

Preparation of Sunitinib Malate-Loaded Lenses (Stabilizer: Caffeine)

1. Lens hydration:

(1) The dry lens to be hydrated prepared in Preparation Example 2 was transferred to a hydration extraction tray (a lid was covered to prevent the lens from flowing out).

(2) According to the formula (i.e. the formula for calculating the volume of hydrated brine: number of lenses×20 mL), the required volume of the hydrated brine was calculated. The hydrated brine was poured into a 5 L beaker and heat to 85±2° C.

(3) After the thermometer showed 85±2° C., the hydration extraction tray was put into the hydration brine and continuously heated for 90 minutes.

(4) After 90 minutes, the hydration extraction tray (including lenses) was taken out and placed in another empty 5 L beaker.

(5) Hydrated saline with the same volume was add into the beaker, and stood at room temperature for 120 minutes to obtain hydrated lenses.

2. Lens loaded with Sunitinib malate:

(1) 200 ppm Sunitinib malate (SM) was prepared and dissolved in saline at 25° C.

(2) 1.3 mL of saline solution (containing 200 ppm SM) was poured into a 5-mL ampoule containing 1 lens. The lens was immersed for 4 hours at room temperature.

(3) The lens preservation solution was replaced with a saline solution containing 150 ppm Sunitinib malate and 0.1 wt % caffeine.

(4) The ampoule was capped and placed in an autoclave for sterilizing at 121° C. for 30 minutes. The overall procedure was about 2 hours, and the drug-loaded lens was obtained after sterilization.

### Example 4

Release of Lens Drug (Sunitinib Malate)

Experimental design:

1. Sampling time point: 0, 2, 4 and 8 hours (N=3)

2. Test groups:

The lens composition includes ethyl methacrylate, ethylene glycol dimethacrylate, methacrylic acid, hydroxyethyl methacrylate and a small amount of photoinitiator, etc. In this example, the ratio of pseudorotaxane to contact lens casting monomer mixture reaches 2.5 wt %.

First group: P-2.5HPC7/P6k-38CL (P-2.5%-38CL)

Second group: P-2.5%-38CL+caffeine (P-2.5%-38CL-Caf)

3. Conditions of drug release:

(1) Single drug-loaded lens was placed in a 5 mL ampoule. 1 mL of simulating tear fluid (STF) (The composition of STF is shown in Table 2 below) was added as a release medium. The lens was placed in a shake oven (MS Hybridization Shaking Oven, MO-AOR, Major Science), and a drug release test was conducted under the operating parameters of 35° C. and 100 rpm. The drug release solution was replaced with fresh STF every one hour.

(2) The drug content in the lens preservation solution and the drug content in the lens were analyzed by HPLC with RP-C18 column. The extraction method of drug (Sunitinib malate, SM) in the lens: one lens was taken out of the preservation solution. Lens surface moisture was fully absorbed with a lens cleaning paper. The one lens was immersed in 1 mL of methanol to stand for 8 hours to extract the drug. Then the drug content was analyzed by HPLC.

TABLE 2
Composition of STF:

| Ingredients | Concentration (g/L) |
|---|---|
| Sodium chloride (NaCl) | 6.78 |
| Sodium bicarbonate (NaHCO₃) | 2.18 |
| Potassium chloride (KCl) | 1.38 |
| Calcium chloride (CaCl₂) | 0.084 |
| albumin | 3.94 |
| mucin | 0.2 |

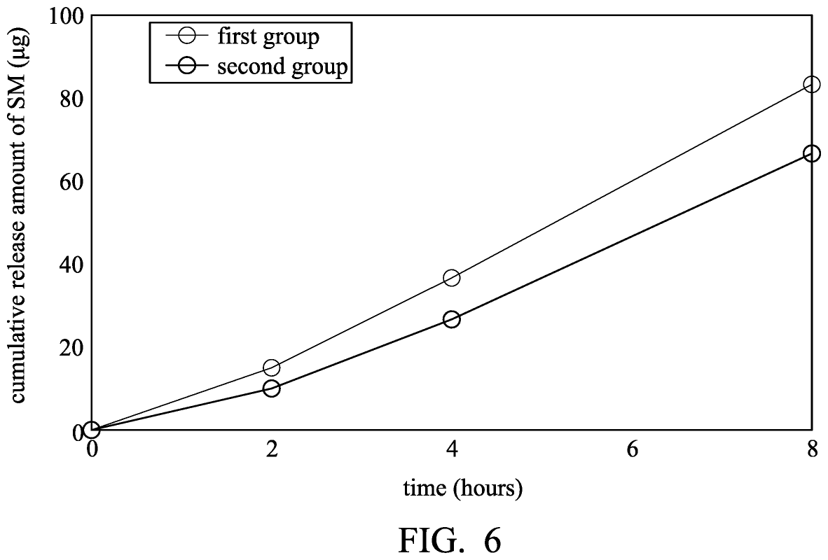

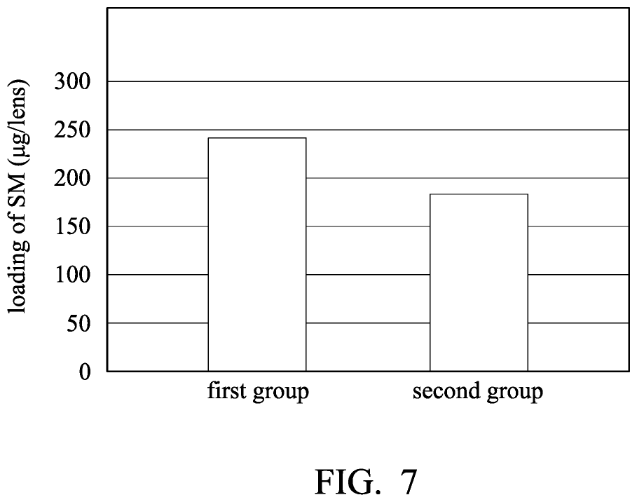

4. Release of lens drug (referring to the following Table 3 and FIGS. 6 and 7):

(1) The loading of Sunitinib malate (SM) in the lens is about 190-230 μg/lens. The addition of caffeine (the lens was immersed in 1 mg caffeine/mL) slightly reduces the drug loading of the lens to SM (as shown in FIG. 7).

(2) In the presence of caffeine, the loss of drug due to the moist heat sterilization is only 3.4 wt %. If there is no caffeine, the moist heat sterilization will cause 9-15 wt % drug loss.

(3) The loading of caffeine in the lens is about 138 μg/lens.

(4) After the lens is loaded with caffeine, the drug release rate is delayed (as shown in FIG. 6).

TABLE 3

| | SM loading | Caf loading |
|---|---|---|
| Lens code | (μg/lens) (N = 3) | (μg/lens) (N = 3) |
| P-2.5%-38CL | 229.57 ± 4.64 | 0 |
| P-2.5%-38CL-Caf | 187.36 ± 5.89 | 137.97 |

While the invention has been described by way of example and in terms of the preferred embodiments, it should be understood that the invention is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A contact lens with a sustained-release property, comprising:
a polymer, polymerized by monomer materials, wherein the monomer materials comprise ethyl methacrylate, ethylene glycol dimethacrylate, methacrylic acid, 2-hydroxyethyl methacrylate, or a combination thereof;
pseudorotaxane, embedded in the polymer; and
a stabilizer, embedded in the pseudorotaxane, wherein the stabilizer comprises surfactants, amino acids with a polar side chain, oligopeptides containing at least one amino acid with a polar side chain, purine or its derivatives, or a combination thereof.

2. The contact lens with a sustained-release property as claimed in claim 1, wherein the pseudorotaxane is composed of cyclodextrin and polyethylene glycol.

3. The contact lens with a sustained-release property as claimed in claim 2, wherein the cyclodextrin comprises α-cyclodextrin, β-cyclodextrin, γ-cyclodextrin, hydroxypropyl-alpha-cyclodextrin, hydroxypropyl-beta-cyclodextrin, hydroxypropyl-gamma-cyclodextrin, sulfobutyl-beta-cyclodextrin, or a combination thereof.

4. The contact lens with a sustained-release property as claimed in claim 2, wherein the polyethylene glycol has an average molecular weight ranging between 2,000 and 20,000.

5. The contact lens with a sustained-release property as claimed in claim 2, wherein the cyclodextrin and the polyethylene glycol have a weight ratio ranging between 0.5:1 and 500:1.

6. The contact lens with a sustained-release property as claimed in claim 1, wherein the pseudorotaxane and the polymer have a weight ratio ranging between 1:4 and 1:200.

7. The contact lens with a sustained-release property as claimed in claim 1, wherein the stabilizer and the pseudorotaxane have a weight ratio ranging between 1:1 and 1:20.

8. The contact lens with a sustained-release property as claimed in claim 1, further comprising an active ingredient embedded in the pseudorotaxane.

9. The contact lens with a sustained-release property as claimed in claim 8, wherein the active ingredient and the pseudorotaxane have a weight ratio ranging between 1:0.1 and 1:40,000.

10. The contact lens with a sustained-release property as claimed in claim 8, wherein the active ingredient in the contact lens has a content ranging between 0.1 μg/lens and 1,000 μg/lens.

11. The contact lens with a sustained-release property as claimed in claim 8, wherein the active ingredient comprises menthol, steroids or their derivatives, antibiotics, tyrosine kinase inhibitors, or a combination thereof.

12. The contact lens with a sustained-release property as claimed in claim 1, wherein the contact lens is further placed in a preservation solution.

13. The contact lens with a sustained-release property as claimed in claim 12, wherein the preservation solution provides the stabilizer.

14. A contact lens used for sustained release of drugs, comprising:
a polymer, polymerized by monomer materials, wherein the monomer materials comprise ethyl methacrylate, ethylene glycol dimethacrylate, methacrylic acid, 2-hydroxyethyl methacrylate, or a combination thereof;
pseudorotaxane, embedded in the polymer; and
a stabilizer, embedded in the pseudorotaxane, wherein the stabilizer comprises surfactants, amino acids with a polar side chain, oligopeptides containing at least one amino acid with a polar side chain, purine or its derivatives, or a combination thereof.
